(19) United States
(12) Patent Application Publication
(10) Pub. No.: US 2024/0065594 A1
(43) Pub. Date: Feb. 29, 2024

(54) SYSTEM AND METHOD FOR NON-INVASIVE MIND READING USING COHERENT RADIO WAVES

(71) Applicant: Diji Nedungayil Jayakaran, Ernakulam (IN)

(72) Inventor: Diji Nedungayil Jayakaran, Ernakulam (IN)

(21) Appl. No.: 18/488,915

(22) Filed: Oct. 17, 2023

(30) Foreign Application Priority Data
Aug. 10, 2023 (IN) 202341053830

(51) Int. Cl. A61B 5/16; A61B 5/00; A61B 5/05

(52) U.S. Cl. CPC A61B 5/16; A61B 5/05; A61B 5/4064; A61B 5/6803; A61B 2503/12; A61B 2560/0223; A61B 2562/066; G06F 3/015

(57) ABSTRACT

The concept behind this invention is to utilize coherent radio waves to detect ion transport in neurons, thereby interpreting thoughts and other brain functions. Radio waves have the property of reflecting from charged ions, while also having the ability to penetrate human tissues. By emitting coherent radio waves and analyzing their reflections, it becomes possible to determine the location of ion transport within specific neurons of the brain. The application of back projection methods further assists in accurately interpreting the exact location of ion reflection. Since different areas of the brain have specific functions, analyzing the location of ion transport in neurons provides insights into various brain activities, such as thoughts and movement of body parts.

### TECHNICAL FIELD

The present invention relates generally to fields of Biomedical engineering/applications and more specifically related to non-invasive brain study using radio waves to provide a detailed picture of brain activities including thoughts.

### BACKGROUND

The invention of “mind reading by coherent radio waves that reflect from ions in ion channels of neurons” is based on a thorough understanding of the electrochemical flow imaging of the brain and other human organs, as well as the properties of radio waves and their interactions with human tissues.

The method begins with the realization that ions, particularly sodium, calcium, and chloride, play crucial roles in the functioning of the brain through their transport in the axons of neurons. As coherent radio waves pass through the brain, these ions, present in ion channels, exhibit behavior such as reflection, refraction, polarization, or absorption of the radio waves.

To conduct the mind-reading process, an array of radio wave sources is employed to generate radio waves in a coherent and synchronized manner. These waves are then directed towards the human brain, and they can easily penetrate non-conducting materials such as the skull and human body. Some radio waves pass through the brain, while others interact with the ions in the neural pathways, causing them to reflect the waves back.

The key aspect of the method lies in the measurement and analysis of the reflected radio waves' output characteristics. By carefully studying the peaks in the electric field produced by these reflections and comparing them with controlled signals generated without the presence of the human body, researchers can deduce the specific paths of ions within the nerves.

This enables a detailed examination of the electrochemical flow in the brain, offering insights into how neural pathways and circuits function during various cognitive processes, emotions, and thoughts. Essentially, it opens the door to decoding brain activity and potentially understanding the neural basis of consciousness.

The inventors emphasize the safety aspects of using radio waves in this context. Unlike ionizing radiation, which can damage DNA and cause harmful effects, radio waves are non-ionizing and do not possess the energy required to produce such effects. Instead, their main impact is to generate heat. However, in this invention's application, the radio waves are carefully calibrated to be within safe limits for human exposure.

U.S. Ser. No. 11/141,088B2 by Sony Corp, describes an electronic device that handles recognition of mental behavioral, affect, emotional, mental states, mental health, or mood-based attributes based on deep neural networks (DNNs), stores a set of EEG signals and a set of bio-signals associated with a subject. The electronic device trains a plurality of first recognition models on a training set of EEG signals and a training set of bio-signals associated with different training subjects. The electronic device trains a second recognition model on a feature vector from output layers of the plurality of first recognition models. The electronic device estimates a plurality of dependency or relationship data by application of the trained plurality of first recognition models on the set of EEG signals and bio-signals. The electronic device identifies a mental behavioral attribute of the subject by application of the trained second recognition model on the plurality of signals and their relationship data.

U.S. Ser. No. 11/144,596B2 by Harman International Industries Inc A method for retrieving information includes detecting a first neural activity of a user, wherein the first neural activity corresponds to a key thought of the user; in response to detecting the first neural activity, generating a search query based on speech occurring prior to detecting the first neural activity; retrieving information based on the search query; and transmitting the information to one or more output devices.

WO2019214899A1 Embodiments provide thought classifier devices that convert analog electroencephalogram signals obtained from mental activity of a person to a digital signal bitstream data; identify a portion of the bitstream as a thought chunk representing discrete thought activity in response to matching, via a first artificial neural network comparison, digital signal bitstream thought chunk portion metadata to metadata labeled in association with a thought within a thoughts data set, the first artificial neural network trained on the thoughts data set; identify a user category in response to matching, via a different, second artificial neural network comparison, metadata of the thought chunk portion to labeled metadata within the thoughts data set, the second artificial neural network trained on the thoughts data set; and identify a specific thought of the thoughts data that has metadata that has corresponding metadata.

US20210212764A1 by Emad N. Eskandar et al Low power MASER (Microwave Amplification by Stimulated Emission of Radiation) radiation is used to non-invasively record molecular activity in a biological object such as a brain. Low power MASER radiation is also used to neuromodulate molecular targets via Rabi coupling, resulting for example in conformational and function change in specific molecular targets such as ligand-gated ion channels, voltage-gated ion channels, G-proteins, or dopamine receptors. The method can be used to change the energy state of targeted molecules via energization or enervation, or to ablate targeted molecules. This method will not be very useful since a microwave can only penetrate 1 or 2 centimeters into the brain. However, in our invention radio waves are used, which will fully penetrate the brain and provides 3d images of complete activities in the brain.

The theoretical background provided further supports the feasibility of the invention. It delves into the physiological basis of electrical signals in neurons, explaining how ion channels and ion transport contribute to depolarization and hyperpolarization of the cell membrane potential. Additionally, the generation of magnetic fields due to the electrical currents in the dendrites and axons are well studied. While the magnetic fields can be relatively strong in the axons, they are still quite weak when measured externally. This helps dismiss the possibility of using local magnetic fields to input sensory information to tubulins, which have been theorized to be involved in consciousness.

### SUMMARY

The present invention overcomes the above-described and other problems and disadvantages in the prior art and provide an innovative solution for non-invasive reading of mind.

The invention presents a sophisticated and non-invasive approach to study brain function and potentially read thoughts by analyzing the electrochemical flow within neurons using coherent radio waves, which are in phase. By measuring the interactions of radio waves with ions in ion channels and carefully analyzing the output characteristics, a user/researcher can gain deep insights into brain activities and potentially unravel the mysteries of human cognition and consciousness.

The invention proposes a novel approach to understand brain activity and potentially read or determine thoughts by using coherent radio waves to probe the ion flow within the neurons of the brain. The basis of the method lies in the fact that ions, such as sodium, calcium, and chloride, present in ion channels within neurons, have the ability to reflect, refract, polarize, or absorb radio waves.

To perform mind-reading technique, an array of radio wave sources is utilized to generate radio waves in phase, which results in diffraction. These waves are then directed towards the human brain, where they penetrate the skull and human body, without causing any harm due to their non-ionizing nature. As these radio waves pass through the brain, some are absorbed, while others interact with the ions in the neural pathways and reflect back.

By measuring the peaks in the electric field produced by these reflected radio waves and comparing them with control signals obtained without the human body, it becomes possible to deduce the path of ions within the neural networks. This information, in turn, can provide insights into how electrochemical flow occurs during different brain activities, potentially revealing patterns related to thoughts, memories, and other cognitive functions.

The inventors take into account the safety concerns associated with radio waves and clarify that they are non-ionizing, meaning they lack the energy to cause ionization or DNA damage. The main effect of radio waves is to generate heat. However, radio waves can penetrate deeper into the body, and their depth of penetration depends on their frequency, material resistivity, and permittivity.

The electrical signals are propagated in neurons through ion channels, leading to depolarization and hyperpolarization of the cell membrane potential are well studied. The Hodgkin-Huxley model is mentioned, which helps to understand the ionic currents involved in generating the neuronal action potential.

The magnetic fields generated by these electric currents in dendrites and axons. The calculated magnetic field strength in axons is found to be stronger than in dendrites due to the greater ion currents flowing inside the axoplasm. However, the magnetic field strengths measured in experiments were relatively weak, ruling out their potential for inputting sensory information to microtubules, if they are indeed involved in consciousness, as hypothesized.

The invention proposes a sophisticated and non-invasive method to study brain function by analyzing ion flow in neurons using coherent radio waves. By measuring the reflection and interaction of radio waves with ions in ion channels, researchers could potentially gain insights into brain activities and decipher aspects of human cognition and consciousness.

The method begins with the realization that ions, particularly sodium, calcium, and chloride, play crucial roles in the functioning of the brain through their transport in the axons of neurons. As coherent radio waves pass through the brain, these ions, present in ion channels, exhibit behavior such as reflection, refraction, polarization, or absorption of the radio waves.

To conduct the mind-reading process, an array of radio wave sources is employed to generate radio waves in a coherent and synchronized manner. These waves are then directed towards the human brain, and they can easily penetrate non-conducting materials such as the skull and human body. Some radio waves pass through the brain, while others interact with the ions in the neural pathways, causing them to reflect the waves back.

The key aspect of the method lies in the measurement and analysis of the reflected radio waves' output characteristics. By carefully studying the peaks in the electric field produced by these reflections and comparing them with controlled signals generated without the presence of the human body, researchers can deduce the specific paths of ions within the nerves.

This enables a detailed examination of the electrochemical flow in the brain, offering insights into how neural pathways and circuits function during various cognitive processes, emotions, and thoughts. Essentially, it opens the door to decoding brain activity and potentially understanding the neural basis of consciousness.

In this invention's application, the radio waves are carefully calibrated to be within safe limits for human exposure. The theoretical background provided further supports the feasibility of the invention. It delves into the physiological basis of electrical signals in neurons, explaining how ion channels and ion transport contribute to depolarization and hyperpolarization of the cell membrane potential.

In another embodiment, the invention presents a sophisticated and non-invasive approach to study brain function and potentially read thoughts by analyzing the electrochemical flow within neurons using coherent radio waves. By measuring the interactions of radio waves with ions in ion channels and carefully analyzing the output characteristics, researchers can gain deep insights into brain activities and potentially unravel the mysteries of human cognition and consciousness.

The embodiment of this concept revolves around utilizing coherent radio waves to detect ion transport in neurons and interpret brain functions in humans and other organisms. Similar to the principle of a laser measuring distance, coherent radio waves are employed to determine the location of ion transport within specific neurons. Back projection methods aid in interpreting the precise location from which the radio waves reflect. As various brain areas are already associated with specific functions, analyzing the location of ion transport in neurons enables the identification of ongoing brain activities, including thoughts and body movements.

Filtered back projection techniques using linear or bilinear interpolation or iterative reconstruction, can be employed to accurately determine the location from which radio waves reflect from ions in ion channels within neurons. By utilizing a scanning radio wave device, the system can analyze and interpret the ion transport patterns to decipher brain activities.

In this concept a headgear with an array of coherent radio wave emitters and detectors are placed on human's head and coherent radio wave emitted from headgear's emitters are reflected from ions moving through in ion channels of neurons. By comparing the reflected and non-reflected radio waves we can find out the exact location of neuron activity to a single neuron or area of neurons where neurons are active. Using back projection method using linear or bilinear interpolation we can analyze and get a 3D model of brain highlighting brain activity. coherent radio waves from multiple directions are sent to brain from headgear and reflected radio waves are analyzed to find out the distance from emitter and where which radio waves are reflected from ions. that distance pinpoints the area from where ion activity happens in brain. Since areas of the brain that are unique to thought, memory etc. are already known we can interpret what activities are happening in the brain. Since individual neuron activity can be mapped, we get a comprehensive picture of what is happening in the brain. Distance measurers work by sending pulses of coherent radio waves. The radio wave reflects off an ion in ion channels of neurons, and the measurer calculates the amount of time it takes for the reflection to return to the device. An internal processor calculates distance based on how long it takes the reflection to return. Or we can use the method where, coherent radio waves is focused through the brain, and data gathered by reading the differences in refracted or reflected radio wave intensities from the brain caused by the charged ions.

Radio waves possess the capability of serving as an electrochemical flow imaging method for the brain and other human organs. High-frequency or shortwave radio waves reflect from charged ions. When a radio wave encounters ions, the electric field within the wave induces oscillation in the electrons of the ions, resulting in resonance at the same frequency as the radio wave. Some of the radio-frequency energy is transferred to this resonant oscillation, and the oscillating electrons may recombine or re-radiate the original wave energy. Total refraction occurs when the collision frequency of the ions is lower than the radio frequency, given a sufficiently high electron density within the ions. The oscillating electric currents generate radio waves.

Ions within the nerves reflect, refract, polarize, or absorb radio waves. As the brain primarily functions through ion transport within neuron axons, passing known radio waves through the brain and analyzing the output characteristics enables the study of electrochemical flow in the brain. Radio waves can penetrate the skull and human body while passing through different environments, experiencing reflection, refraction, polarization, diffraction, and absorption. Various frequencies encounter different combinations of these phenomena within the Earth's atmosphere, making certain radio bands more suitable for specific purposes than others.

Radio waves interact with charged particles by accelerating or decelerating charges. Accelerating charged particles emit electromagnetic waves because it has to conserve momentum. That Electromagnetic wave depends on acceleration of the particle, the larger the acceleration greater the EM pulse. Emitted EM wave is tangential to motion of charged particle. This is regulated by Lienard's law for relativistic version, non-relativistic approximation gives rise to Larmor's radiation law based on Maxwell's laws for electromagnetism. By analyzing emitted EM waves from ions in the brain we can locate the ion activity in brain. By interpreting this ion activity in the brain, we can find what activity is happening in brain.

Radio waves can effectively penetrate non-conductive materials, such as wood, bricks, and concrete and human tissues. However, they cannot pass through electrical conductors like water or metals. In this proposed method, an array of radio wave sources emits radio waves in phase, causing diffraction. While some radio waves pass through the human body, others get absorbed, and ions within nerves reflect these radio waves. By measuring peaks in the electric field and comparing them with sources and detectors without a human body, it becomes possible to determine the path of ions within nerves for every fraction of a second.

Neuronal electrical signals primarily occur through ion channels. Positive ions, such as sodium and calcium, entering the cytoplasm led to depolarization of the membrane, while negative ions like chloride induce hyperpolarization. The flow of ions from the extracellular space towards the cytoplasm is called influx, while the opposite flow is termed efflux. Specific ion channels and ion pumps extrude ions from the cytoplasm to the extracellular space, with positive ion efflux (potassium) leading to hyperpolarization and negative ion efflux (hydrogen carbonate) resulting in depolarization. Normal neuronal membrane potentials vary from approximately −90 mV to +50 mV. Neuronal membrane ion channels are typically ligand or voltage gated, responding to neuromediator molecules or changes in membrane potential, respectively.

The primary communication between two neurons occurs through axo-dendritic synapses located at the top of dendritic spines, which are characteristic of cortical neurons. Dendritic postsynaptic membranes convert neuromediator signals into postsynaptic electric currents. Neuromediator molecules bind to specific postsynaptic ion channels, opening their gates and allowing ion flux. The ion species entering the dendritic cytoplasm alter the membrane potential. If multiple dendritic inputs are temporally and spatially summated, it becomes possible to measure axial dendritic voltages in the range of tens of millivolts. Consequently, the electric intensity along the dendritic axis in different regions of the dendritic tree could reach up to 10 V/m.

The proposed invention combines the principles of radio wave technology, neuroscience, and advanced signal processing algorithms to unlock new horizons in the field of cognitive science. By harnessing the power of coherent radio waves and analyzing ion transport in neurons, this technology offers ground-breaking possibilities in mind reading and brain function analysis.

### BRIEF DESCRIPTION OF THE DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

The diagrams are for illustration only, which thus is not a limitation of the present disclosure, and wherein:

FIG. 1 illustrates a wearable helmet with coherent radio wave emitters and detectors, in accordance with a preferred embodiment of the invention.

FIG. 2 illustrates the absorption and penetrating capacity of electromagnetic waves of different frequency through the human body, in accordance with a preferred embodiment of the invention.

FIG. 3 illustrates the system comprises a computer system, in accordance with a preferred embodiment of the invention.

FIG. 4 illustrates the detailed drawing of a neuron, in accordance with a preferred embodiment of the invention.

FIG. 5 illustrates a schematic diagram of how reflected and non-reflected radio waves pass through brain from emitter to detector, in accordance with a preferred embodiment of the invention.

FIG. 6 illustrates a schematic diagram of human subject with head gear and computing system to analyze and interpret data and a display unit to view brain activities like thoughts, emotions, memory motor commands from brain, etc.

### DETAILED DESCRIPTION

Embodiments of the present disclosure include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and firmware or by human operators.

Various terms as used herein are shown below. To the extent a term used in a claim is not defined below, it should be given the broadest definition persons in the pertinent art have given that term as reflected in printed publications and issued patents at the time of filing.

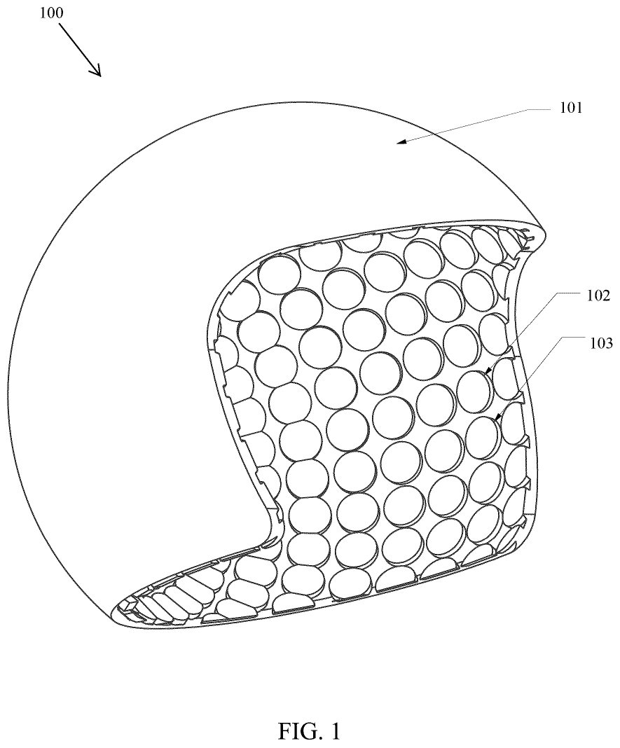

FIG. 1 illustrates a non-invasive system 100 comprised of a head gear with coherent radio wave emitters and detectors. As illustrates a head gear casing 101 with coherent radio wave emitters 102 and radio wave detectors 103. The headgear casing 101 with an array of coherent radio wave emitters 102 and detectors 103 are placed on human's head and coherent radio wave that are in phase emitted from headgear are reflected from ions moving through in ion channels of neurons. By comparing the reflected and non-reflected radio waves we can find out the exact location of neuron activity to a single neuron or area of neurons where neurons are active. Using back projection method using linear or bilinear interpolation we can analyze and get a 3D model of brain highlighting brain activity. The coherent radio waves from multiple directions are sent to brain from headgear and reflected radio waves are analyzed to find out the distance from emitter and where which radio waves are reflected from ions. that distance pinpoints the area from where ion activity happens in brain.

Since areas of the brain that are unique to thought, memory etc. are already known we can interpret what activities are happening in the brain. Since individual neuron activity can be mapped, we get a comprehensive picture of what is happening in the brain. Since neuron level details of ion activities can be detected by this device, we can pinpoint exact location of neurons with activity and interpret results to find out what thoughts are happening and what action is being initiated and what are inside the memory areas of brain. Distance measurers work by sending pulses of coherent radio waves. The radio waves reflects off an ion in ion channels of neurons, and the measurer calculates the amount of time it takes for the reflection to return to the device. An internal processor calculates distance based on how long it takes the reflection to return. Or we can use the method where coherent radio waves is focused through the brain, and data gathered by reading the differences in refracted or reflected radio wave intensities from the brain caused by the charged ion.

### Headgear Casing and Array of Coherent Radio Wave Emitters and Detectors:

The headgear casing is designed with precision and comfort in mind. It consists of a lightweight, non-conductive material that can be easily adjusted to fit different head sizes. The array of coherent radio wave emitters and detectors is integrated into the headgear at strategic positions to ensure optimal coverage of the brain's surface.

### Coherent Radio Wave Generation:

The coherent radio wave emitters use advanced electronics and signal processing techniques to generate high-frequency, low-power radio waves with a well-defined phase and amplitude. These waves are typically in the short-wave radio frequency range, which allows them to penetrate the brain's tissue while maintaining their coherence.

Interaction with Neural Tissue:

When the headgear is placed on a human head, the coherent radio waves emitted from the array of emitters penetrate the scalp and skull, reaching the brain tissue. As the radio waves pass through the brain, they encounter ions present in the ion channels of active neurons. These ions, which are charged particles, cause scattering and reflection of the radio waves.

### Reflection and Detection:

The coherent radio wave detectors within the headgear continuously monitor the radio waves' reflections. The detectors are equipped with sensitive antennas and receivers capable of capturing the reflected radio waves with high fidelity. Additionally, they measure the phase and amplitude changes of the reflected waves, which are indicative of the ion activity and distances traveled.

### Distance Measurement:

To determine the distance from the emitter to the ion channel causing the reflection, the system uses time-of-flight measurements. The coherent radio waves travel at the speed of light, and by calculating the time it takes for the reflected wave to return to the detector, the distance can be precisely estimated.

### Data Acquisition and Pre-processing:

The system collects a vast amount of data from the coherent radio wave reflections. To handle this data, specialized algorithms are employed for pre-processing and noise reduction. Calibration procedures ensure accurate measurement and calibration of the detectors to account for variations in the radio wave propagation through the brain tissue.

### Back Projection Method and 3D Brain Activity Model:

The pre-processed data is then subjected to a back projection method, where the reflected radio waves are “back-projected” into the brain volume. By combining the information from multiple detectors and emitters, the system creates a 3D model of the brain's activity, highlighting regions with active neurons.

### Spatial Resolution Enhancement:

To improve spatial resolution, the system uses interpolation techniques such as linear or bilinear interpolation. This helps to refine the 3D model and allows for a more accurate representation of neuron activity at specific locations.

### Interpretation of Brain Activity:

Known brain regions associated with particular cognitive functions serve as a reference for interpreting the 3D brain activity model. By identifying regions with heightened neuron activity, the system can deduce the type of cognitive process occurring in those areas, such as thinking, memory retrieval, or motor planning.

### Multi-Directional Radio Wave Emission:

The system employs multiple-directional radio wave emission to obtain a more comprehensive view of brain activity. By sending coherent radio waves from different angles and analyzing the reflected waves, the accuracy of localization and the robustness of the brain activity model are improved.

This technical description explores a novel approach to non-invasive mind reading, utilizing radio waves to detect ion activity in the brain. The theory is based on the interaction between radio waves and charged particles, wherein accelerating charged particles emit electromagnetic waves. By analyzing the emitted electromagnetic waves from brain ions, it becomes possible to locate ion activity and interpret brain activity. This technique offers promising applications in brain-computer interfaces and cognitive research, although challenges related to sensitivity and spatial resolution must be addressed.

Non-invasive mind reading has the potential to revolutionize various fields, from brain-computer interfaces to cognitive neuroscience. This technical description presents a theoretical framework that utilizes radio waves to detect ion activity in the brain, enabling remote monitoring and interpretation of brain function.

### Alternative Measurement Method:

Interaction of Radio Waves and Charged Particles, Radio waves, being a form of electromagnetic radiation, interact with charged particles in their vicinity. When a charged particle is accelerated or decelerated, it emits electromagnetic waves as it seeks to conserve momentum. The emitted electromagnetic wave's amplitude and characteristics depend on the magnitude of acceleration, leading to a larger electromagnetic pulse for greater acceleration. To describe the behavior of the emitted electromagnetic waves, we employ Lienard's law for the relativistic version and Larmor's radiation law for the non-relativistic approximation. Lienard's law is based on Maxwell's laws for electromagnetism and governs the tangential emission of electromagnetic waves with respect to the motion of the charged particle.

Radio Wave Detection System, consists of three main components:
a. Radio Wave Emitter: A low-power, frequency-modulated radio wave emitter is placed in close proximity to the subject's head or within specialized headgear. It emits radio signals in a specific frequency range.
b. Radio Wave Detector: A sensitive radio wave receiver is used to pick up weak electromagnetic signals emitted by ions in the brain. The detector must have a wide bandwidth to capture signals in the desired frequency range.
c. Signal Processing Unit: A powerful signal processing unit processes the received radio wave signals to extract meaningful information related to brain activity.

When the brain is active, ion movements within neural tissue generate varying electric fields, leading to the acceleration and deceleration of charged particles. These accelerated charges emit weak electromagnetic waves that propagate through the skull and scalp, where they can be detected by the radio wave detector. A short pulse of EM waves will decelerate ions moving against it, after EM wave is cut off ions start accelerating. These accelerating ions emit EM waves. By analyzing this delayed emission of radio waves, we can locate the exact location of ion activities in the brain. The captured radio wave signals undergo pre-processing to filter out noise and artifacts. Advanced signal processing techniques, such as Fourier transforms and wavelet analysis, are applied to extract meaningful features from the data. By analyzing the emitted electromagnetic waves, it becomes possible to determine the location of ion activity in the brain. Different brain regions and cognitive processes can be associated with distinct patterns of ion activity, allowing for localization and interpretation of brain function.

Radio waves interact with charged particles by accelerating or decelerating charges. Accelerating charged particles emit electromagnetic waves because it has to conserve momentum. That Electromagnetic wave depends on acceleration of the particle, the larger the acceleration greater the EM pulse. Emitted EM wave is tangential to motion of charged particle. This is regulated by Lienard's law for relativistic version, non-relativistic approximation gives rise to Larmor's radiation law based on Maxwell's laws for electromagnetism. By analyzing emitted EM waves from ions in the brain we can locate the ion activity in brain. By interpreting this ion activity in the brain, we can find what activity is happening in the brain.

Interpreting Brain Activity, the detected ion activity can be interpreted to understand various cognitive states and thoughts. During calibration, the subject performs specific mental tasks or is exposed to stimuli, and the corresponding brain activity and emitted electromagnetic wave patterns are recorded. Machine learning models can then be trained to correlate specific patterns with known brain activities.

### Alternative Measurement Method:

In addition to the time-of-flight based distance measurement, the system can also use a refractometry-based approach. Here, coherent radio waves are focused through the brain, and data on the differences in refracted or reflected radio wave intensities from the brain's charged ions are gathered.

The concept of reading the human mind using coherent radio waves and advanced signal processing techniques opens up a new frontier in neuroscience research and brain-computer interfaces. By accurately mapping individual neuron activity and creating detailed 3D brain activity models, this technology holds immense potential for understanding the complexities of the human brain and developing innovative applications in the fields of medicine, psychology, and neuroscience.

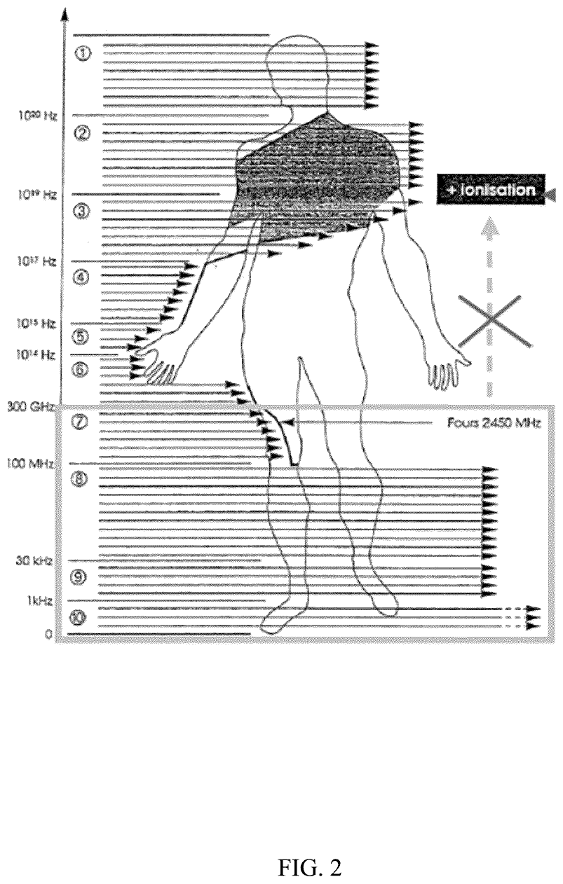

FIG. 2 illustrates the absorption and penetrating capacity of electromagnetic waves of different frequency through the human body, in accordance with a preferred embodiment of the invention. Different frequency rays penetrate the human body, and the absorption and penetrating capacity of electromagnetic waves is different for different frequency through human body. Cosmic rays, Gamma rays, X-ray, Ultraviolet, Visible Spectrum, Infrared, Microwave, radio wave, Lower frequencies and ELF are illustrated on the chart and numbers respectively like 1 for Cosmic rays, 2 for Gamma rays and so on.

In FIG. 2, the numerical labels correspond to various types of electromagnetic radiation as follows: 1 denotes cosmic rays, 2 represents gamma rays, 3 signifies x-rays, 4 stands for ultraviolet rays, 5 pertains to the visible spectrum, 6 is associated with infrared radiation, 7 relates to microwave radiation, 8 corresponds to radio waves, 9 designates low frequencies, and 10 is allocated to extremely low frequencies (ELF). Within the figure, the shaded area within the human body indicates the presence of ionizing radiation, specifically, gamma rays (labeled as 2) and x-rays (labeled as 3). It is noteworthy that microwaves (labeled as 7) possess the ability to penetrate the human body to a limited depth, typically a few centimeters.

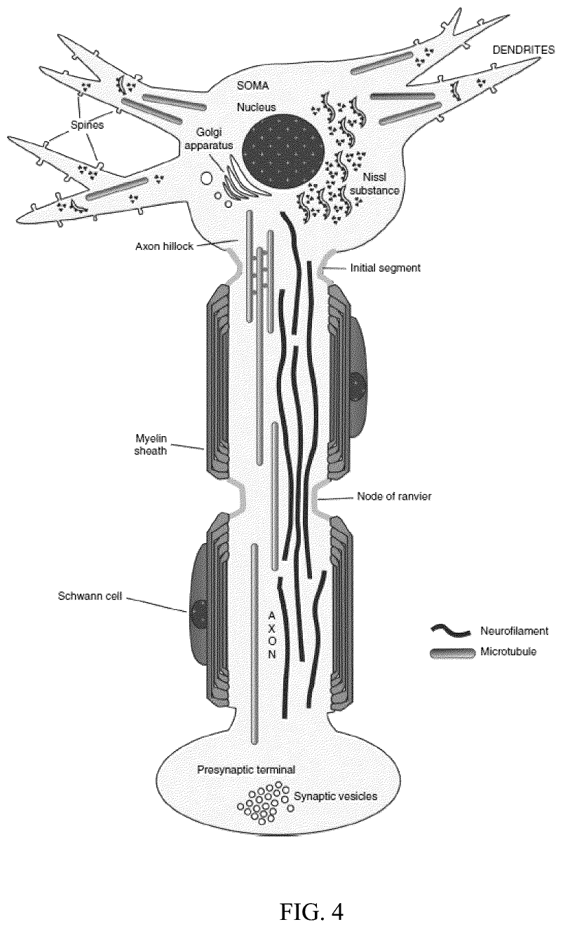

Coherent radio waves are directed towards the human brain from multiple emitters in headgear, and they can easily penetrate non-conducting materials such as the skull and human body. Some radio waves pass through the brain, while others interact with the ions in the neural pathways, causing them to reflect the waves back. To perform this mind-reading technique, an array of radio wave sources is utilized, generating radio waves in phase, which results in diffraction. These waves are then directed towards the human brain, where they penetrate the skull and human body, without causing any harm due to their non-ionizing nature. To conduct the mind-reading process, an array of radio wave sources is employed to generate radio waves in a coherent and synchronized manner. These waves are then directed towards the human brain, and they can easily penetrate non-conducting materials such as the skull and human body. Some radio waves pass through the brain, while others interact with the ions in the neural pathways, causing them to reflect the waves back as shown in FIG. 4. The key aspect of the method lies in the measurement and analysis of the reflected radio waves' output characteristics. By carefully studying the peaks in the electric field produced by these reflections and comparing them with controlled signals generated without the presence of the human body, researchers can deduce the specific paths of ions within the nerves.

Mind reading through coherent radio waves reflecting from ions in ion channel in neurons analysis is a concept that involves using a headgear casing equipped with an array of coherent radio wave emitters and detectors to measure brain activity. The emitted coherent radio waves are reflected from ions moving through ion channels in neurons. By analyzing the reflected and non-reflected radio waves, it becomes possible to determine the exact location of neuron activity, down to a single neuron or specific areas where neurons are active. This description will provide a detailed technical explanation of the process involved in this mind-reading technique.

The headgear casing (101) is a wearable device that houses an array of coherent radio wave emitters (1022) and detectors (103). The emitters are responsible for transmitting coherent radio waves into the brain, while the detectors pick up the reflected waves. The placement of the headgear is critical to ensure accurate and consistent readings. It should be properly positioned on the human head, and the emitters and detectors must be aligned correctly.

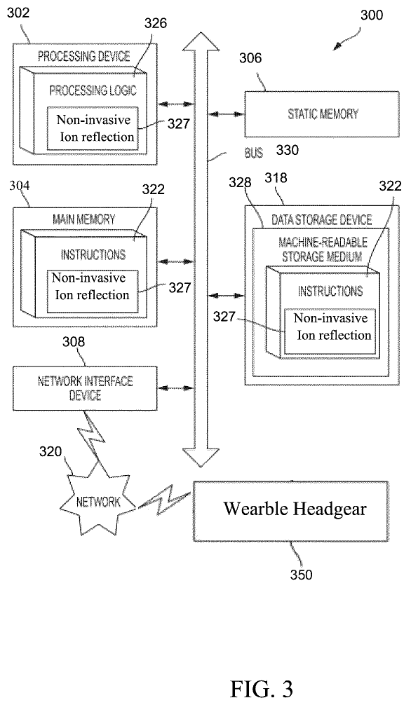

FIG. 3 illustrates a mind reading using a system using a computer system 300 within which a set of instructions, for causing the machine to perform any one or more of the methodologies discussed herein, may be executed. In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 300 may be representative of a server computer system, such as system 100.

The exemplary computer system 300 includes a processing device 302, a main memory 304 (e.g., read-only memory (ROM), flash memory, dynamic random-access memory (DRAM), a static memory 306 (e.g., flash memory, static random-access memory (SRAM), etc.), and a data storage device 318, which communicate with each other via a bus 330. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Processing device 302 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 302 may also be one or more special-purpose processing devices such as an application specific integrated circuit (ASIC), a field programmable gate array (FPGA), a digital signal processor (DSP), network processor, or the like. The processing device 302 is configured to execute processing logic 326 comprising Non-invasive Ion reflection (327), which may be one example of system 100 shown in FIG. 1, for performing the operations and steps discussed herein.

The data storage device 318 may include a machine-readable storage medium 328, on which is stored one or more set of instructions 322 (e.g., software) embodying any one or more of the methodologies of functions described herein, including instructions to cause the processing device 302 to execute system 100. The instructions 322 may also reside, completely or at least partially, within the main memory 304 or within the processing device 302 during execution thereof by the computer system 300; the main memory 304 and the processing device 302 also constituting machine-readable storage media. The instructions 322 may further be transmitted or received over a network 320 via the network interface device 308.

The machine-readable storage medium 328 may also be used to store instructions to perform the methods and operations described herein. While the machine-readable storage medium 328 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

A Wearable headgear (340) is connected to the computer system through a network interface or a wireless network.

Coherent radio waves are electromagnetic waves with a fixed phase relationship between their oscillation.

FIG. 4 illustrates the detailed drawing of a neuron, in accordance with a preferred embodiment of the invention.

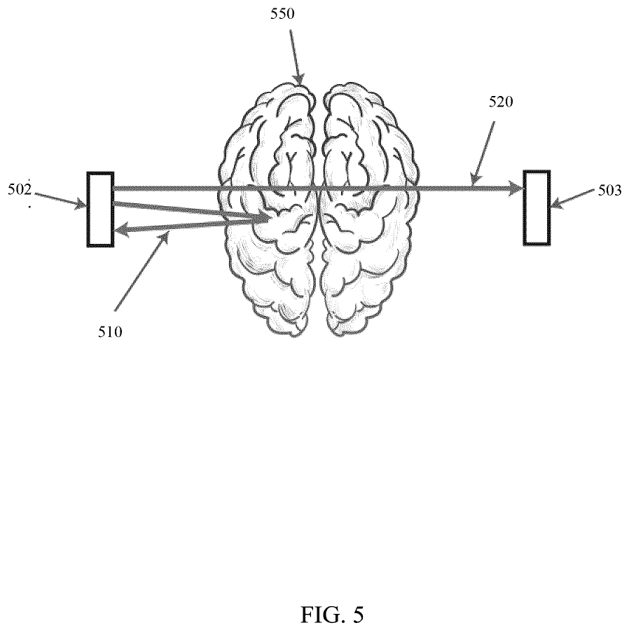

FIG. 5 illustrates a schematic diagram of how reflected and non-reflected radio waves pass through the brain (550) from emitter (502) to detector (503). The reflected radio waves are reflected (510) from a portion of the brain and there are some radio waves which are not reflected (520) and pass through the brain.

The time taken by the reflected waves to come back to the emitter or detector will help in calculating or determining the position of the brain from where the waves are reflected back and helps in understanding the though process of the user.

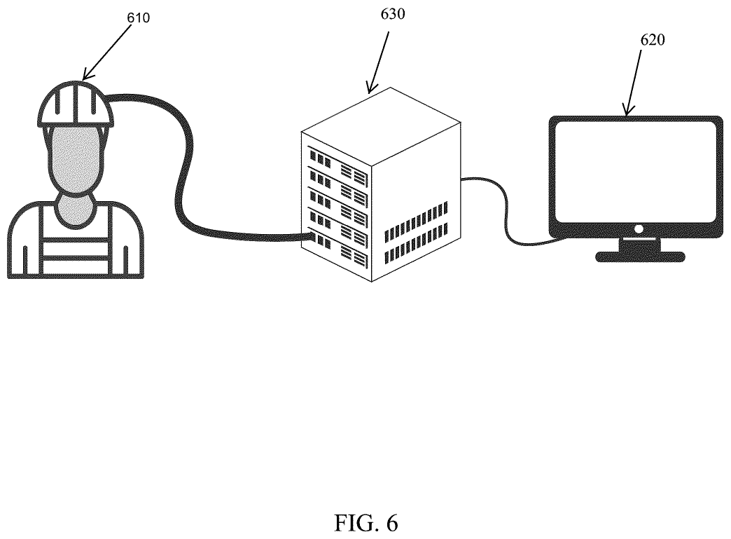

FIG. 6 illustrates a schematic diagram of human subject with head gear (610) and computing system (630) to analyze and interpret data and a display unit (620) to view brain activities like thoughts, emotions, memory motor commands from brain, etc.

A computing system (630) comprises of a processing device representing one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing devices may also be one or more special-purpose processing devices such as an application specific integrated circuit (ASIC), a field programmable gate array (FPGA), a digital signal processor (DSP), network processor, or the like. The processing device is configured to execute processing logic, which may be one example of system shown in FIG. 6, for performing the operations and steps discussed herein.

These waves have the ability to penetrate biological tissues, including the human skull and brain. The headgear emits coherent radio waves towards the brain in a controlled manner.

When coherent radio waves encounter ions moving through ion channels of neurons, the waves interact with these charged particles. This interaction results in the scattering and reflection of the radio waves. The reflected waves carry information about the ion activity within the neurons.

The reflected radio waves are collected by the detectors in the headgear. By analyzing the time delay between the emitted and reflected waves, the system can calculate the distance between the emitter and the point of reflection. This distance measurement is crucial for pinpointing the location of ion activity in the brain.

To obtain a comprehensive 3D model of brain activity, the back projection method is utilized. This technique involves tracing the paths of the reflected radio waves backward from the detectors to the brain. By employing linear or bilinear interpolation, the system can reconstruct a 3D representation of the brain, highlighting areas of neuron activity.

As different areas of the brain are known to be associated with specific functions like thoughts, memories, and actions, the 3D model's analysis allows for the interpretation of brain activity. By correlating the detected neuron activity with the known functions of brain regions, it becomes possible to understand what thoughts are occurring, what actions are being initiated, and even delve into the contents of memory areas.

One of the key advantages of this mind-reading technique is the ability to detect individual neuron activity. By precisely pinpointing the exact location of neurons with activity, the system can provide a detailed and comprehensive picture of what is happening in the brain at the neuron level.

Since the technology allows for mapping neuron activity in real-time, it enables a comprehensive view of brain activity during various tasks, experiences, and cognitive processes. This level of granularity can offer invaluable insights into brain function and help in understanding neurological disorders and cognitive phenomena.

The concept of mind reading using coherent radio waves reflecting from ions in ion channel reflections in neurons is a theoretical approach to understand and interpret brain activity non-invasively. It draws inspiration from the principles of compact disc (CD) reading by lasers, where a laser beam interacts with a CD's surface, and the reflected light carries encoded information. Similarly, coherent radio waves are focused through the brain, and the differences in refracted or reflected radio wave intensities, caused by charged ions in the ion channels of neurons, are measured and interpreted to gain insights into brain activity and cognitive processes.

Coherent radio waves are electromagnetic waves that have a consistent phase relationship and amplify one another when combined. In this mind reading method, coherent radio waves are generated and focused on the brain non-invasively. The frequencies of these radio waves must be carefully chosen to ensure minimal interference with the brain's natural electrical activity while still penetrating the brain tissue effectively.

Neurons, the fundamental building blocks of the brain, communicate through electrical impulses generated by the flow of charged ions across their cell membranes. These ions, such as sodium (Na+), potassium (K+), and calcium (Ca2+), play a crucial role in initiating and propagating neural signals. Ion channels, specialized proteins embedded in the cell membrane, control the movement of these ions in and out of the neuron.

When coherent radio waves pass through brain tissue, they encounter the ion channels present in the neurons. The charged ions within the ion channels interact with the electric component of the radio waves, causing changes in the refractive index and altering the reflection properties of the radio waves.

Sophisticated imaging and sensing equipment are utilized to measure the refracted or reflected radio wave intensities after they interact with the brain. This data contains complex patterns and variations corresponding to the distribution and activity of ion channels in the brain regions being examined.

The gathered data consists of intricate patterns that need to be decoded and interpreted to extract meaningful information about brain activity. Advanced signal processing techniques, such as Fourier transforms and time-domain analysis, are employed to identify specific patterns and correlations.

The next step involves mapping the recorded radio wave intensity patterns to the corresponding brain regions. This requires accurate spatial localization and precise mapping techniques to associate specific patterns with distinct brain functions or cognitive processes.

Decoding the brain activity entails relating the measured changes in ion channel reflection or refraction to specific neural processes. This process is highly complex, as the brain's functioning is influenced by a multitude of factors, and correlations between brain activity and behavior or cognitive states must be established through extensive research and analysis.

In an exemplary aspect, this innovative method aims to decode cognitive thoughts by harnessing synchronized, phase-aligned radio waves to monitor ion transport within neurons and analyze brain activity. It involves a series of well-defined steps to achieve this goal:
Monitoring Brain Activity: The process begins with the monitoring of brain activity. When a person engages in cognitive tasks, various regions of the brain become active. Within these regions, ion movements occur within neural tissue. These ion movements lead to the creation of variable electric fields.
Generating Electromagnetic Waves: The electric fields generated by ion movements induce the acceleration and deceleration of charged particles within the neural tissue. As a result of these dynamic processes, charged particles emit weak electromagnetic waves. These waves, though subtle, have the unique property of propagating through both the skull and the scalp.
Detection of Electromagnetic Waves: A critical element of this method is the use of a specialized radio wave detector. This detector is capable of capturing the emitted electromagnetic waves as they pass through the skull and scalp. It effectively “listens” to these waves, which carry information about the brain's ion activity.
Pulse of Electromagnetic Waves: To further enhance the process, a brief pulse of electromagnetic waves is introduced within the neural tissue. This pulse serves to decelerate ions that are moving in opposition to it.
Delayed Emission of Electromagnetic Waves: Once the pulse of electromagnetic waves is cut off, the ions that were decelerated begin to accelerate again. This renewed acceleration of ions results in the emission of additional electromagnetic waves. Importantly, these emitted waves are delayed compared to the initial pulse.
Precise Localization of Ion Activities: The heart of the method lies in the analysis of these delayed emissions of radio waves. By carefully examining the characteristics of these emissions, it becomes possible to precisely localize the areas of ion activity within the brain. This provides valuable insights into which parts of the brain are actively engaged during specific cognitive tasks.
Data Preprocessing: To ensure the accuracy of the results, the collected radio wave signals undergo preprocessing. This step involves filtering out any extraneous noise or artifacts that may have been picked up during the detection process.
Advanced Signal Processing: To extract meaningful information from the data, advanced signal processing techniques are employed. These include mathematical tools like Fourier transforms and wavelet analysis, which are used to identify relevant features in the collected data.
Interpreting Brain Function: By analyzing the emitted electromagnetic waves and the patterns of ion activity they reveal, researchers can determine the specific locations of ion activity within the brain. Different brain regions and cognitive processes are associated with distinct patterns of ion activity, enabling the localization and interpretation of various aspects of brain function.

In essence, this method offers a sophisticated means of deciphering cognitive thoughts by tapping into the subtle electromagnetic signals generated by ion movements in the brain, providing valuable insights into the intricate workings of the human mind.

Areas of Applications
Enhanced Communication: This breakthrough technology will enable individuals with communication impairments, such as those with locked-in syndrome or neurodegenerative disorders, to express their thoughts and needs effortlessly. It opens up new avenues for inclusive and accessible communication.
Neuroscience Research: By enabling researchers to access real-time neural activity without invasive methods, our technology will revolutionize the field of neuroscience. This non-invasive approach could accelerate advancements in brain-computer interfaces, cognitive psychology, and the study of neurological disorders.
Crime investigation and Court Proceedings: this concept can be used to read the thoughts of accused and witnesses to find out the truth easily. This will help the government to employ law enforcement very easily and crime rates will fall to a very low level.
Human machine or computer interface: you can control machines like cars and other devices by just your thoughts.
Creativity and Arts: you can use this device to read the mind and create the designs or music that are generated in your brain. You don't need to study how to draw or how to compose music, to create new design or music, you just have to think about a design or particular music and this device can reproduce that design or music on a computer.
Gaming and Entertainment: Integration of mind reading technology into gaming and entertainment platforms could redefine the user experience, creating immersive virtual environments and interactive narratives. This innovation has the potential to revolutionize the gaming industry and offer unparalleled levels of engagement. You can control games using your thoughts.

While some embodiments of the present disclosure have been illustrated and described, those are completely exemplary in nature. The disclosure is not limited to the embodiments as elaborated herein only and it would be apparent to those skilled in the art that numerous modifications besides those already described are possible without departing from the inventive concepts herein. All such modifications, changes, variations, substitutions, and equivalents are completely within the scope of the present disclosure. The inventive subject matter, therefore, is not to be restricted except in the protection scope of the appended claims.

1. A system to determine the mind thoughts using coherent radio waves, which are in phase to detect ion transport in neurons to analyze brain activity comprises:
a headgear with an array of coherent radio wave emitters and detectors placed on human's head and coherent radio wave emitted from headgear are reflected from ions moving through in ion channels of neurons;
a processor to compare the reflected and non-reflected radio waves to identify the exact location of neuron activity to a single neuron or area of neurons where neurons are active;
determine the distance from the emitter to the ion channel causing the reflection, using time-of-flight measurements;
calculate the time it takes for the reflected wave to return to the detector;
combine the information from multiple detectors and emitters; and
creates a 3D model of the brain's activity, highlighting regions with active neurons known as brain regions associated with particular cognitive functions serve as a reference for interpreting the 3D brain activity model.

2. The system of claim 1, wherein by identifying regions with heightened neuron activity, the system can determine the type of cognitive process occurring in those areas, such as thinking, memory retrieval, or motor planning.

3. The system of claim 1, wherein the system is used to detect ion transport in all biological organisms.

4. The system of claim 1, wherein the system reads brain activities in vertebrates using radio waves to detect ion transport in their brains.

5. The system of claim 1, wherein the system reads the mind using radio waves to detect ion transport in brain for human machine interface for creating art and music and controlling other devices like driving car.

6. The system of claim 1, wherein the system reads mind in using radio waves to detect ion transport in brain for accessing thoughts and memory of the accused and witnesses for crime investigation and court proceedings.

7. The system of claim 1, wherein the system reads brain activities in vertebrates using radio waves to detect ion transport in brain for neuroscience and other medical research.

8. The system of claim 1, wherein system reads mind using radio waves to detect ion transport in brain for the enhancement of human communication ability and for helping physically disabled to perform tasks using thoughts in brain.

9. A method to determine the mind thoughts using coherent radio waves to detect ion transport in neurons to analyze brain activity comprises:
detecting coherent radio wave emitted from headgear are reflected from ions moving through in ion channels of neurons using a headgear with an array of coherent radio wave emitters and detectors placed on human's head;
comparing the reflected and non-reflected radio waves to identify the exact location of neuron activity to a single neuron or area of neurons where neurons are active;
determining the distance from the emitter to the ion channel causing the reflection, using time-of-flight measurements;
calculating the time, it takes for the reflected wave to return to the detector;
combining the information from multiple detectors and emitters; and
creating a 3D model of the brain's activity, highlighting regions with active neurons, known brain regions associated with particular cognitive functions serve as a reference for interpreting the 3D brain activity model.

10. The method of claim 9, wherein the method consisting of a refractometry-based approach by analyzing, difference in intensities between transmitted wave, reflected wave and refracted wave to find the location of reflected or refracted spot to locate activities in neuron in the brain.

11. A method for determining cognitive thoughts by utilizing coherent, phase-aligned radio waves to detect ion transport in neurons and analyze brain activity, comprising the steps of:
monitoring brain activity, where active brain regions exhibit ion movements within neural tissue, and radio waves EM field that accelerate and decelerate charged particles like ions in neurons;
observing the emission of weak electromagnetic waves as a result of the acceleration of charged particles, which propagate through the skull and scalp;
detecting these emitted electromagnetic waves using a radio wave detector;
initiating a short pulse of electromagnetic waves within the neural tissue, causing deceleration of ions moving against it;
observing the subsequent acceleration of these ions and their emission of electromagnetic waves after the electromagnetic pulse is terminated;
analyzing the delayed emission of radio waves to pinpoint the precise location of ion activities within the brain;
subjecting the collected radio wave signals to preprocessing to eliminate noise and artifacts;
employing advanced signal processing techniques such as Fourier transforms and wavelet analysis to extract meaningful data features;
utilizing the analysis of emitted electromagnetic waves to determine the specific locations of ion activity in the brain;
associating distinct patterns of ion activity with different brain regions and cognitive processes, enabling the localization and interpretation of brain functions including thoughts.

12. The method of claim 11, wherein the introduction of the brief pulse of electromagnetic radio waves is precisely synchronized with the ongoing neural activity in real-time, ensuring optimal alignment of the pulse with the ion movements, thus enhancing the accuracy and temporal resolution of ion activity localization within the brain.

13. The method of claim 11, further comprising the step of calibrating the radio wave detector to differentiate between various electromagnetic wave frequencies emitted by ions, enabling the distinction of different types of ion activity and the subsequent localization and categorization of specific cognitive processes within the brain.

14. The method of claim 11, wherein the method by identifying regions with heightened neuron activity, type of cognitive process occurring in those areas, such as thinking, memory retrieval, or motor planning is configured to be determined.

15. The method of claim 11, wherein method is used to detect ion transport in all biological organisms.

16. The method of claim 11, wherein the method reads brain activities in vertebrates using radio waves to detect ion transport in their brains.

17. The method of claim 11, wherein reading mind using radio waves to detect ion transport in brain for human machine interface for creating art and music and controlling other devices like driving car.

18. The method of claim 11, wherein reading the mind in using radio waves to detect ion transport in brain for accessing thoughts and memory of the accused and witnesses for crime investigation and court proceedings.

19. The method of claim 11, wherein reading brain activities in vertebrates using radio waves to detect ion transport in brain for neuroscience and other medical research.

20. The method of claim 11, wherein reading mind using radio waves to detect ion transport in brain for the enhancement of human communication ability and for helping physically disabled to perform tasks using thoughts in brain.
